(19) United States
(12) Patent Application Publication
(10) Pub. No.: US 2024/0139492 A1
(43) Pub. Date: May 2, 2024

(54) DISINFECTING CAP FOR IV CATHETER LUER HUB

(71) Applicant: Becton, Dickinson and Company, Franklin Lakes, NJ (US)

(72) Inventor: Kevin M. Ryan, Whitehouse Station, NJ (US)

(21) Appl. No.: 17/978,599

(22) Filed: Nov. 1, 2022

(51) Int. Cl. A61M 39/16; A61L 2/18; A61L 2/26; A61M 25/00; A61M 39/20

(52) U.S. Cl. CPC A61M 39/162; A61L 2/18; A61L 2/26; A61M 25/0097; A61M 39/20; A61L 2202/122; A61L 2202/123; A61L 2202/24

(57) ABSTRACT

A disinfecting cap engageable with a luer hub of an IV catheter assembly includes a housing defining an inner cavity, with the housing having a top wall, a sidewall extending down from the top wall and with a threaded inner surface, an open bottom end formed by the sidewall and defining an opening, and a tapered protrusion extending downward from the top wall, with the top wall, the sidewall, and the tapered protrusion defining the inner cavity. The disinfecting cap also includes an annular-shaped absorbent disinfecting member disposed within the housing adjacent an inner surface of the top wall and positioned about the tapered protrusion, with the absorbent disinfecting member including a disinfecting solution configured to disinfect portions of the luer hub when attached thereto. The tapered protrusion is configured to interfit with the luer hub to seal off the IV catheter assembly.

### BACKGROUND OF THE INVENTION

### Field of the Invention

The present disclosure relates generally to caps for medical connectors and, in particular, to a disinfecting cap configured to be attached directly to a luer hub of an IV catheter, for sealing and disinfecting portions of the hub.

### Description of Related Art

Catheters are commonly used to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

If not properly maintained or if exposed to a non-sterile environment, a VAD can become contaminated, sealed with blood clots, and/or can spread infection. Further, bacteria and other microorganisms may gain entry into a patient's vascular system from access hubs, ports, or valves upon connection to the VAD to deliver a fluid or pharmaceutical to a patient. Therefore, each access hub (or port/valve or connection) of a VAD or that is configured for attachment to a VAD is associated with some risk of transmitting a catheter related bloodstream infection (CRBSI) to a patient.

In order to decrease CRBSI cases and to ensure VADs are used and maintained correctly, many medical facilities implement sterile practices and protocols to ensure that VADs and access hubs or ports are used properly and do not become sealed or infected. These protocols often include sterilizing the access hubs, ports, and VADs, including requiring that access hubs, ports, and valves be capped with disinfecting caps when not in use, to prevent microbial ingress into the hub, port, or valve and to sterilize areas of the hub, port, or valve of the VAD. Disinfecting caps are disposable cap devices that contain an amount of cleaning or disinfecting solution for sterilizing portions of the port, hub, and valve.

In many VADs, when utilizing an IV catheter, a needleless connector is used to close off the VAD (i.e., to seal off a hub or port of the VAD), with the needleless connector then subsequently accessed to administer medication or other necessary fluids (via the catheter) to the patient. In such arrangements, the needleless connector must be kept clean and sterilized to prevent microbial ingress and possible CRBSI. Most, if not all, disinfecting caps are thus designed to interfit with such needleless connectors, with the disinfecting caps configured to mate with a male and/or female luer connection provided on the needleless connector. As the needless connectors are themselves designed to seal off the fluid path of the VAD (alone or in combination with a clamping device), the disinfecting caps are typically constructed as simply cap-like structures that do not provide any sealing capability when coupled to the needleless connector i.e., they do not seal the IV catheter fluid path.

Accordingly, it is desired to provide a disinfecting cap that is capable of sealing the IV catheter fluid path in a VAD device. Providing a disinfecting cap with such a sealing capability would eliminate the need for both a needleless connector and separate disinfecting cap for the needleless connector, as is currently required with existing disinfecting caps.

### SUMMARY OF THE INVENTION

Provided herein is a disinfecting cap engageable with a luer hub of an IV catheter assembly. The disinfecting cap includes a housing defining an inner cavity, with the housing further including a top wall, a sidewall extending down from the top wall and having a threaded inner surface configured to interlock with a mating feature of the luer hub, an open bottom end formed by the sidewall and defining an opening configured to receive the luer hub, and a tapered protrusion extending downward from the top wall, with the top wall, the sidewall, and the tapered protrusion defining the inner cavity. The disinfecting cap also includes an annular-shaped absorbent disinfecting member disposed within the housing adjacent an inner surface of the top wall and positioned about the tapered protrusion, the absorbent disinfecting member including a disinfecting solution configured to disinfect portions of the luer hub. The tapered protrusion is configured to interfit with the luer hub to seal off the IV catheter assembly.

In some embodiments, the absorbent disinfecting member is a compressible sponge or open cell foam, and wherein the sponge or open cell foam is compressed upon engaging of the disinfecting cap with the luer hub, to release at least some of the disinfecting solution upon being compressed.

In some embodiments, the tapered protrusion has a length less than a length of the sidewall.

In some embodiments, a peel-off protective cover is positioned over the open bottom end of the housing.

In some embodiments, the housing is a single-molded part formed of a rigid thermoplastic polymer.

In some embodiments, the tapered protrusion is a hollow protrusion.

In some embodiments, the luer hub is a female luer connector including a threaded outer surface that engages the threaded inner surface of the sidewall.

In some embodiments, the disinfecting member is configured to clean and/or disinfect the threaded outer surface of the female luer connector.

In some embodiments, the female luer connector defines a tapered cavity with which the tapered protrusion is interfit to seal off the IV catheter assembly.

Also provided is an IV catheter assembly including a catheter hub having a distal end and a proximal end, a catheter coupled to the distal end of the catheter hub and extending out distally therefrom, so as to be positionable intravenously within a patient, an extension line coupled to the proximal end of the catheter hub, and a luer hub positioned at a proximal end of the extension line, the luer hub in fluid communication with the catheter through the extension line and the catheter hub. The IV catheter assembly also includes a disinfecting cap configured to mate with the luer hub, with the disinfecting cap further including a housing comprising a top wall, a sidewall extending down from the top wall and having a threaded inner surface, an open bottom end formed by the sidewall and defining an opening configured to receive the luer hub, and a tapered protrusion extending downward from the top wall, with the top wall, the sidewall, and the tapered protrusion defining an inner cavity within the housing. The disinfecting cap also includes an annular shaped absorbent disinfecting member disposed within the housing adjacent an inner surface of the top wall and positioned about the tapered protrusion, with the absorbent disinfecting member including a disinfecting solution configured to disinfect portions of the luer hub. The tapered protrusion is configured to interfit with the luer hub to seal off the IV catheter assembly.

In some embodiments, the luer hub is a female luer connector including a hub body having a threaded outer surface configured to engage the threaded inner surface of the sidewall and a tapered cavity formed in the hub body and configured to mate with the tapered protrusion to seal off the IV catheter assembly.

In some embodiments, the threaded inner surface of the sidewall includes a single-start thread and the threaded outer surface of the female luer connector includes a double-start thread defining two thread grooves.

In some embodiments, the single-start thread engages a first thread groove of the threaded outer surface and a second thread groove of the threaded outer surface remains open, with the second thread groove forming a fluid channel through which the disinfecting solution can flow when the female luer connector is coupled with the disinfecting cap.

In some embodiments, the threaded inner surface of the sidewall includes a double-start thread and the threaded outer surface of the female luer connector includes a double-start thread.

In some embodiments, the absorbent disinfecting member is a compressible sponge or open cell foam, wherein the sponge or open cell foam is compressed upon engaging of the disinfecting cap with the luer hub, to release at least some of the disinfecting solution upon being compressed.

In some embodiments, the disinfecting cap further includes a peel-off protective cover positioned over the open bottom end of the housing.

In some embodiments, a bottom edge of the sidewall includes a flange formed thereon, with the peel-off protective cover sealed on the flange.

In some embodiments, the tapered protrusion has a length less than a length of the sidewall, such that the tapered protrusion is vertically spaced apart from the open bottom end.

In some embodiments, the tapered protrusion extends to a bottom edge of the sidewall, such that the tapered protrusion is flush with the open bottom end.

In some embodiments, the tapered protrusion is a hollow protrusion.

### BRIEF DESCRIPTION OF THE DRAWINGS

FIG. 1 is a perspective view of an IV catheter assembly, according to an aspect of the present disclosure;

FIG. 1A is a detailed perspective view of a disinfecting cap and luer hub included in the IV catheter assembly of FIG. 1;

FIG. 2 is a perspective views of a disinfecting cap for an IV catheter luer hub, according to an aspect of the disclosure;

FIG. 3 is an exploded perspective view of the disinfecting cap of FIG. 2;

FIG. 4 is a front top perspective view of the housing of the disinfecting cap of FIG. 2;

FIG. 5 is a front bottom perspective view of the housing of the disinfecting cap of FIG. 2;

FIG. 6 is a cross-sectional view of the disinfecting cap of FIG. 2;

FIG. 7 is a cross-sectional view of a disinfecting cap, according to another aspect of the disclosure;

FIG. 8 is a cross-sectional view of the disinfecting cap of FIG. 2 prior to being connected to a luer hub; and

FIG. 9 is a cross-sectional view of the disinfecting cap of FIG. 2 after being connected to a luer hub.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with another component or with a patient. Thus, for example, the end of a device first contacting another component or the body of the patient would be the distal end, while the opposite end of the device being manipulated by the user would be the proximal end of the device.

The terms “first”, “second”, and the like are not intended to refer to any particular order or chronology, but refer to different conditions, properties, or elements.

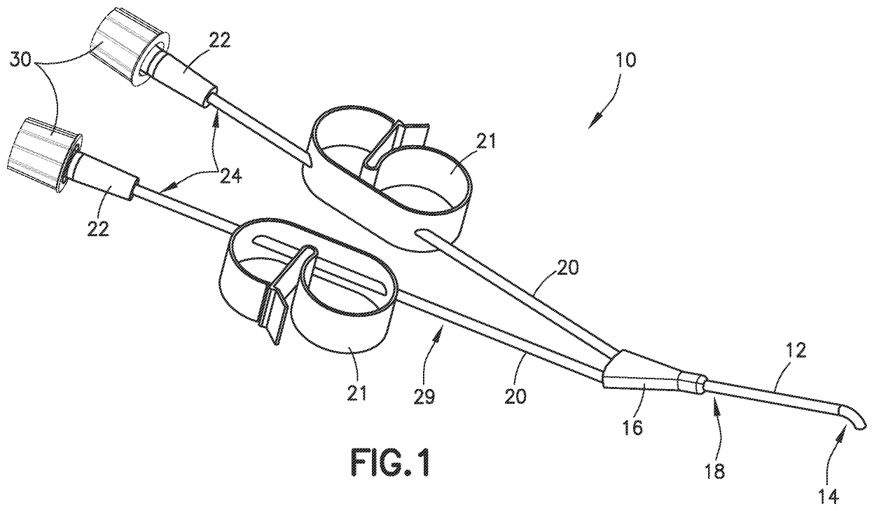

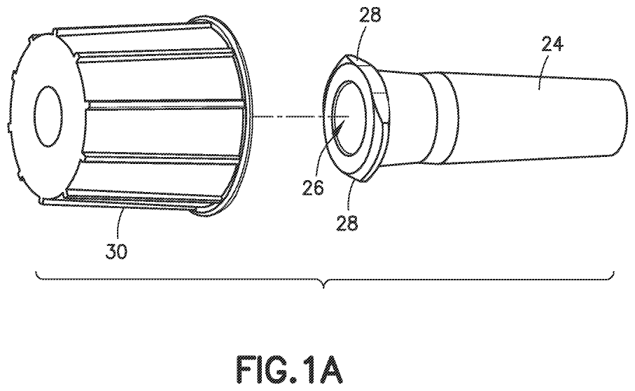

Reference is first made to FIG. 1, which depicts an intravenous (IV) catheter assembly 10 with which aspects of the disclosure may be implemented, according to a non-limiting embodiment. The IV catheter assembly 10 includes a catheter tube 12 having a distal end 14 that may be inserted transcutaneously through the skin of a patient at an insertion site. The IV catheter assembly 10 also includes a bifurcation hub 16 coupled to a proximal end 18 of the catheter tube 12 and a plurality of extension legs 20 that operably connect, via the bifurcation hub 16, to a corresponding number of lumens defined by the catheter tube 12. In some embodiments, a clamp 21 may be provided on each extension leg 20 to control a flow of fluid therethrough by selectively sealing off the extension leg 20.

Each of the extension legs 20 includes a luer hub 22 positioned at a proximal end 24 thereof. As used herein, a “luer” hub refers to a connector that includes a tapered portion (i.e., a luer taper) for creating a friction engagement between a tapered stem or elongated member of a male luer connector and a tapered cavity. Each luer hub 22 is configured as a female luer connection having a tapered cavity 26 configured to receive and engage a tapered stem or elongated member of a male luer connector, as well as a threaded outer surface 28 configured to engage threads on the inner surface of an annular shield of the male luer connector. The female luer connection (tapered cavity 26 and threaded outer surface 28) of each luer hub 22 may be configured according to ISO80369-7 and may, for example, be structured to have a cavity with a 6% taper and with threads on an external surface with a width at a crest of each thread of from about 0.3 mm to about 1.0 mm and a width at a root of the thread from about 0.5 mm to about 1.2 mm.

As shown in FIG. 1, after at least an initial use of the IV catheter assembly 10 and at a time when the IV catheter assembly 10 is not presently being utilized for administering fluids into or out of the body, a disinfecting cap 30 may be coupled to each of the luer hubs 22 to disinfect the hubs and prevent the hubs and an IV catheter fluid path 29 (i.e., fluid path through hubs 22, extension legs 20, bifurcation hub 16, and catheter tube 12) from being contaminated by, for example, microbes, debris, or other contaminants. In addition to disinfecting the luer hubs 22, the disinfecting caps 30 also function to seal off the luer hubs 22 and the IV catheter fluid path 29 upon mating with the luer hubs 22, as will be explained in greater detail below.

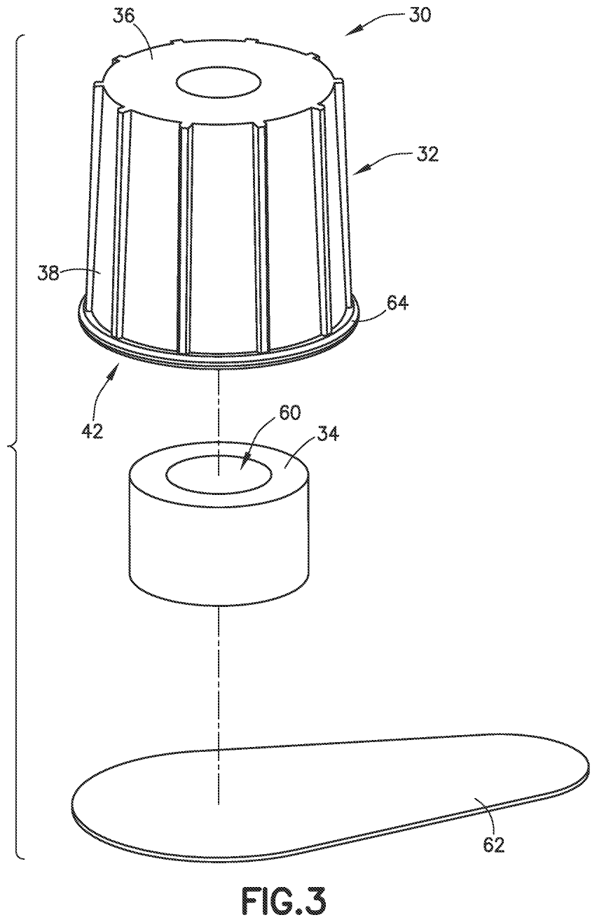

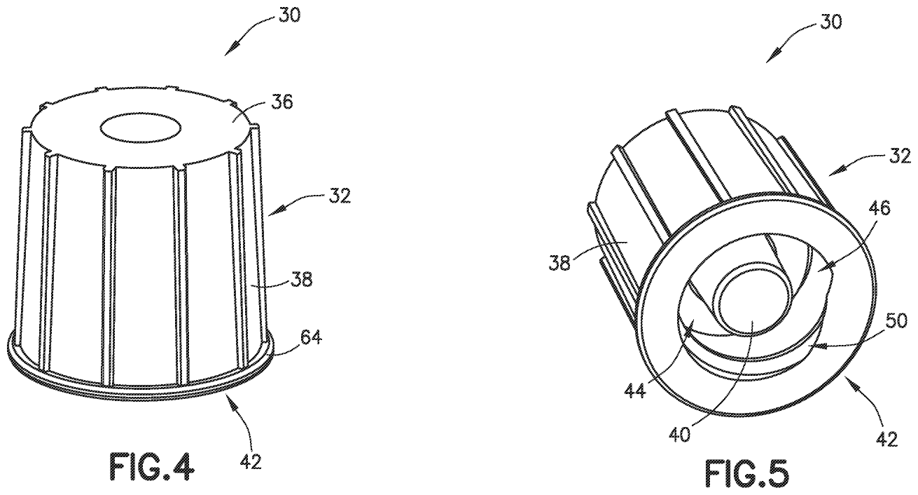

Referring now to FIGS. 2-6, a disinfecting cap 30 is shown in greater detail, along with coupling thereof to an associated hub, in accordance with aspects of the disclosure. The disinfecting cap 30 may, for example, mate with a luer hub 22 of the IV catheter assembly 10 of FIG. 1. The disinfecting cap 30 includes a housing 32 within which is positioned an absorbent disinfecting member 34 having a disinfecting solution contained therein that is configured to disinfect portions of the luer hub 22. As will be explained in further detail below, upon mating of the disinfecting cap 30 with a luer hub 22, the absorbent disinfecting member 34 releases disinfecting solution therefrom that is applied onto portions of the luer hub 22, to thereby disinfect and sterilize the luer hub 22.

As shown in FIGS. 2-9, the housing 32 of disinfecting cap 30 may be formed as a generally cylindrical member that includes a top wall 36, a sidewall 38, a protrusion 40 extending down from the top wall 36, and an open bottom end 42 formed by the sidewall 38. According to embodiments, the protrusion 40 is integrally formed with the top wall 36 and sidewall 38, so as to provide a single, unitary housing 32 that may be easily manufactured. For example, the housing 32 can be formed from a thermoplastic polymer material, such as polyester, polycarbonate, polypropylene, polyethylene, polyethylene terephthalate, or acrylonitrile butadiene styrene, such that the housing 32 may be formed via an injection molding process, according to some embodiments.

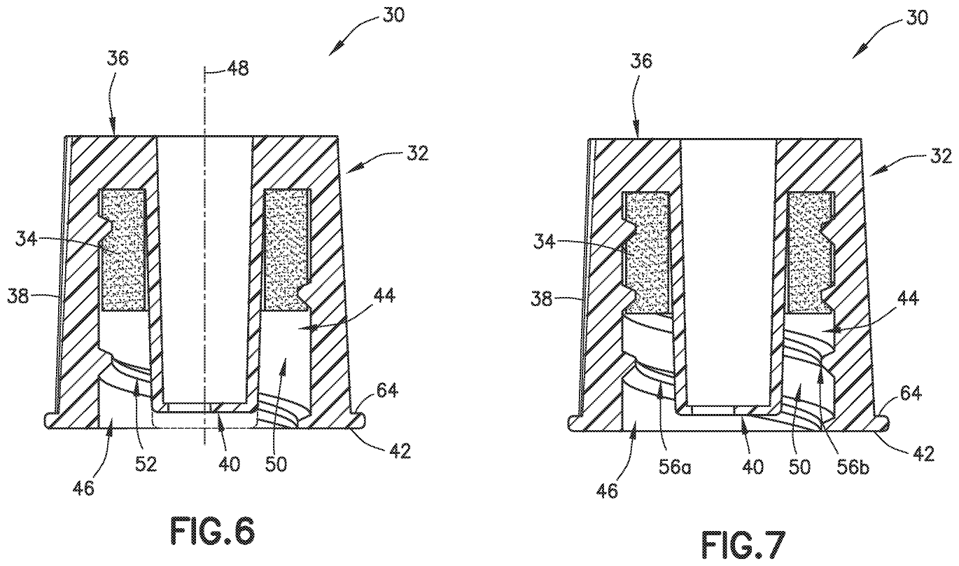

The protrusion 40 of housing 32 may be configured as a tapered member (i.e., a luer taper) configured to interfit with a tapered cavity of a female luer connection, such as of the tapered cavity 26 of luer hub 22. The protrusion 40 may further be configured as a hollow member, with the exterior thereof defining a hollow portion of the protrusion 40. The hollow protrusion 40 may reduce the amount of material required to manufacture the disinfecting cap 30, although it is recognized that the protrusion 40 may instead be constructed as a solid member. Additionally, the protrusion 40 may have a height that is less than that of the sidewall 38, such that the protrusion 40 does not extend all the way down to the open bottom end 42 of housing 32. In other embodiments, the protrusion 40 could extend down so as to be flush with the open bottom end 42, as shown in phantom in FIG. 6.

The housing 32 is configured such that the top wall 36, sidewall 38, and protrusion 40 collectively define or form an inner cavity 44 of the housing 32, with the open bottom end 42 of the housing 32 presenting an opening 46 to the cavity 44 that provides for positioning of the luer hub 22 within the cavity 44. The shape of the top wall 36 and sidewall 38, along with the extending of protrusion 40 down from the top wall 36 at a location aligned with a central axis 48 of the disinfecting cap 30, results in the cavity 44 having a generally annular shape. The disinfecting cap 30 is thus recognized as being structured as a male luer connection, with the protrusion 40 providing a tapered stem of the connection and the sidewall 38 (that is spaced apart from protrusion 40) providing an annular shield extending about the tapered protrusion 40.

According to aspects of the disclosure, the sidewall 38 of the housing 32 has a threaded inner surface 50 that engages and interlocks with the threaded outer surface or segment 28 of the luer hub 22. In order to secure the disinfecting cap 30 to the luer hub 22, the disinfecting cap 30 is twisted relative to the luer hub 22, so as to cause the corresponding threaded surfaces 50, 28 to engage and draw the luer hub 22 together with the disinfecting cap 30.

In one embodiment, the threaded inner surface 50 of the sidewall 38 of disinfecting cap 30 is provided as a single-start thread 52, while the threaded outer surface 28 of luer hub 22 is provided as a double or multi-start thread 54: According to embodiments, the threaded outer surface 28 of luer hub 22 may define a first thread groove and a second thread groove thereon, and may have a profile according to ISO80369-7, FIG.B.5, ISO80369-7, FIG.B.6, or similar, which are incorporated herein by reference. Thus, when mating the luer hub 22 with the disinfecting cap 30, a first thread groove of the threaded outer surface 28 of luer hub 22 may engage the single-start thread 52 on the housing 32 of disinfecting cap 30, and the luer hub 22 may be drawn into the cavity 44 of housing 32 of disinfecting cap 30 by twisting the luer hub 22 relative to the disinfecting cap 30. Meanwhile, while the first thread groove engages the single-start thread 52, the second thread groove of the threaded outer surface 28 of luer hub 22 remains open, with no thread mating therewith. The open second thread groove on threaded outer surface 28 of luer hub 22 may, as explained in further detail below, serve as a fluid channel through which disinfecting solution may flow when the disinfecting cap 30 is coupled to the luer hub 22, while also preventing pressurizing of the disinfecting fluid during coupling.

In another embodiment, and as shown in FIG. 7, the threaded inner surface 50 of the sidewall 38 of disinfecting cap 30 is provided as a double-start thread 56 and the threaded outer surface of luer hub 22 is provided as a double-start thread groove. When mating the luer hub 22 with the disinfecting cap 30, the thread grooves of the double-start thread groove on threaded outer surface 28 of luer hub 22 may engage the threads 56a, 56b of the double-start thread 56 on the housing 32 of disinfecting cap 30, and the luer hub 22 may be drawn into the cavity 44 of housing 32 of disinfecting cap 30 by twisting the luer hub 22 relative to the disinfecting cap 30.

In mating the disinfecting cap 30 with the luer hub 22, and as the luer hub 22 is drawn into the cavity 44 of housing 32 of disinfecting cap 30, the tapered protrusion 40 of housing 32 is positioned within the tapered cavity 26 of luer hub 22. Upon full engagement of the disinfecting cap 30 with the luer hub 22, the tapered protrusion 40 is interit with the tapered cavity 26 to form a tight seal therewith, thereby closing or sealing entry into the IV catheter assembly fluid path 29 (i.e., fluid path through extension legs 20, hub 16, and catheter tube 12). The seal formed between protrusion 40 and tapered cavity 26 of luer hub 22 prevents disinfecting solution from entering into the IV catheter assembly fluid path 29, but also seals the IV catheter assembly fluid path 29 from the external environment to prevent contaminants and/or bacteria from entering into the IV catheter assembly fluid path 29.

As previously described, disinfecting cap 30 includes a disinfecting member 34 therein configured to disinfect portions of the luer hub 22. In particular, disinfecting member 34 can be configured to disinfect surfaces of a proximal end 58 of the luer hub 22, including the threaded outer surface 28 formed thereon, so as prevent microbial ingress and minimize chances of CRBSI.

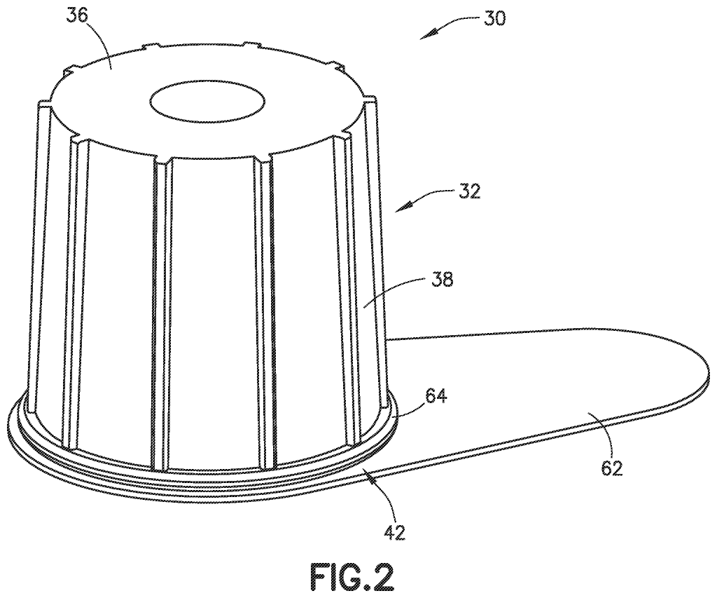

As shown in FIGS. 3 and 6-9, the disinfecting member 34 is an annular structure positioned within the cavity 44 of the housing 32. The disinfecting member 34 is positioned about the protrusion 40 of housing 32, with the protrusion 40 inserted through an opening 60 in the disinfecting member 34. The disinfecting member 34 is held in an upper portion of the cavity 44, such that the disinfecting member 34 may be positioned adjacent the top wall 36 of the housing 32, with the disinfecting member 34 extending down to fill a portion of the cavity 44. In some embodiments, the disinfecting member 34 may be held in position within the cavity 44 via contact thereof with the threaded inner surface 50 of the sidewall 38, with the thread(s) 52 on sidewall 38 keeping the absorbent material 50 under radial compression.

The disinfecting member 34 can be formed from an absorbent material capable of absorbing a cleaning or disinfecting solution for cleaning and/or disinfecting portions of the luer hub 22. In some examples, the disinfecting member 34 can comprise a porous foam (e.g., an open cell foam) or sponge capable of absorbing the cleaning or disinfecting solution. For example, the foam material can be a Plastazote® foam, which is an engineered polymer foam by Zotefoams PCL. In other examples, the foam material can be a polyurethane foam, as known in the art, including an open cell structure capable of absorbing a disinfectant or cleaning solution. In other embodiments, the material of the disinfecting member 34 can be abrasive with sufficient texture, friction, and/or anti-slip properties to scrub surfaces of the luer hub 22 to mechanically remove microbes, debris, and other contaminants from surfaces of the luer hub 22. For example, the disinfecting member 34 can comprise a thermoplastic elastomer, such as polypropylene, polyethylene, or synthetic or natural rubber (e.g., isoprene).

The disinfecting member 34 can be provided (i.e., presoaked) with the cleaning or disinfecting solution. For example, the cleaning or disinfecting solution can be an antimicrobial, anti-fungal, antibacterial, or antiviral solution that sterilizes surfaces of the luer hub 22. In some examples, the cleaning solution can be isopropyl alcohol (IPA), such as about 70% IPA. In other examples, the cleaning solution can be about 0.5% to about 3.5% chlorhexidine gluconate in combination with about 70% EPA. A chlorohexidine composition may be beneficial because it has a slower evaporation rate than IPA and, therefore, provides a more persistent disinfectant activity after the disinfecting cap 30 is removed from the luer hub 22 and before an additional connector (e.g., needleless connector) and associated component of a vascular access device (VAD) is connected to the luer hub 22.

As the disinfecting member 34 is formed from a porous foam, sponge, or other elastomeric material, the disinfecting member 34 is configured as compressive member. Accordingly, when the disinfecting cap 30 is mated with the female connection of luer hub 22, the luer hub 22 may compress the disinfecting member 34 when drawn into the cavity 44 via mating of the threaded outer surface 28 of luer hub 22 with the threaded inner surface 50 of housing 32. Compression of the disinfecting member 34 releases cleaning or disinfecting solution from the disinfecting member 34, with it being recognized that the amount of disinfecting solution released from the disinfecting member 34 may be controlled based on how much the disinfecting member 34 is compressed (based on an initial height/thickness of the disinfecting member 34) and the amount of cleaning or disinfecting solution contained therein. The cleaning or disinfecting solution contacts portions of the luer hub 22, such as the proximal end 58 and threaded outer surface 28, for disinfecting the luer hub 22. As previously described, the engaging of the single thread 52 on inner surface 50 of housing 32 with a first thread groove of threaded outer surface 28 of luer hub 22 can guide disinfecting fluid through an open second thread groove on threaded outer surface 28 of luer guide 22, so as to direct disinfecting solution along a desired path (away from tapered cavity 26 of luer hub 22) and prevent pressurizing of the disinfecting fluid during coupling of the disinfecting cap 30 and luer hub 22.

As shown in FIGS. 2 and 3, the disinfecting cap 30 can further comprise a removable and/or disposable (i.e., peel-off) protective cover 62. The peel-off protective cover 62 is positioned over the open bottom end 42 of the housing 32 and may be attached to a flange 64 formed on the bottom edge of sidewall 38. In an embodiment where the protrusion 40 extends down to be flush with the open bottom end 42, the peel-off protective cover 62 may also couple to the protrusion 40. The peel-off protective cover 62 is provided to protect components and portions of the disinfecting cap 30, such as the cavity 44 of the housing 32 and the disinfecting member 34. In particular, the peel-off protective cover 62 can protect the disinfecting cap 30 during transport and storage to prevent contamination and to prevent the cleaning or disinfecting solution from evaporating prior to use. The peel-off protective cover 62 can comprise a sheet, such as a polymer film, with adhesive on a first side of the sheet for removably mounting the peel-off protective cover 62 to the open bottom end 42 of the housing 12. Alternatively, the peel-off protective cover 62 can be removably mounted to the open bottom end 42 of the housing 32 by heat sealing. The peel-off protective cover 62 can be formed from a material that is impervious or substantially impervious to air, so that the cleaning or disinfecting solution on the disinfecting member 34 does not evaporate or dry-out prior to use of the disinfecting cap 30. Accordingly, the peel-off protective cover 62 can increase a shelf life of the disinfecting cap 30, as well as prevent microbes and other debris from collecting in the disinfecting cap 30 prior to use.

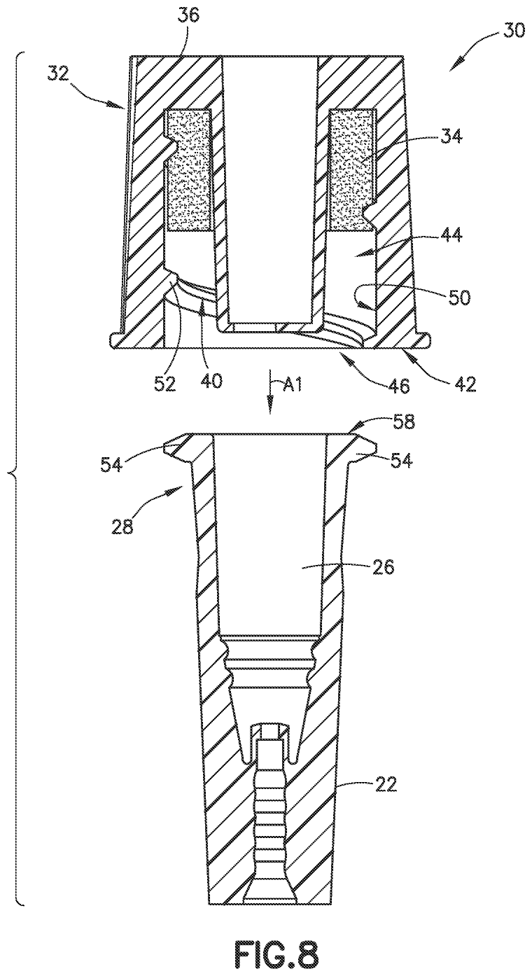

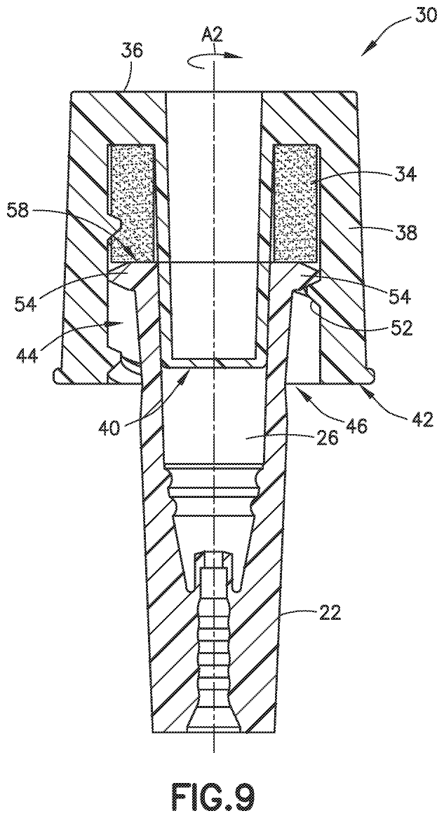

Reference is now made to FIGS. 8 and 9 to describe a process for connecting the disinfecting cap 30 to a luer hub, such as the luer hub 22 shown in FIG. 1. First, the practitioner removes any packaging from the disinfecting cap 30 and removes the protective cover 62 from the open bottom end 42 of the housing 32. The practitioner then moves the disinfecting cap 30 toward the luer hub 22, as shown by arrow A1 in FIG. 8, causing the threaded outer surface 28 of the luer hub 22 to contact/engage the threaded inner surface 50 of the housing 32. The practitioner then rotates the housing 32 relative to the luer hub 22, as shown by arrow A2 in FIG. 9, causing the proximal end 58 of the luer hub 22 to be drawn into the housing 32. Drawing the luer hub 22 into the housing 32 causes the protrusion 40 of the disinfecting cap 30 to be positioned in the tapered cavity 26 of the luer hub 22 and form a seal therebetween. Additionally, drawing the luer hub 22 into the housing 32 causes the luer hub 22 to contact a bottom surface of the disinfecting member 34, which may compress the disinfecting member 34, thereby releasing disinfecting solution from the disinfecting member 34. The disinfecting solution contacts portions of the luer hub 22, such as the proximal end portion 58 and threaded outer surface 28 of the luer hub 22 for cleaning and disinfecting the luer hub 22. As previously described, the engaging of single-start thread 52 on housing 32 with a double-start thread 54 on luer hub 22 can guide disinfecting fluid through an open channel groove of the luer hub 22, to direct the disinfecting solution along a desired path.

In order to remove the disinfecting cap 30 from the luer hub 22, the practitioner rotates the disinfecting cap 30 in an opposite direction (i.e., an opposite direction of arrow A2 in FIG. 9), causing the threaded inner surface 50 of the housing 32 to release from the threaded outer surface 28 of the luer hub 22. Once released, the practitioner can pull the disinfecting cap 30 away from the luer hub 22. With the disinfecting cap 30 removed from the luer hub 22, a VAD can then be connected to the IV catheter assembly 10 through the luer hub 22, as previously described.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

1. A disinfecting cap engageable with a luer hub of an intravenous (IV) catheter assembly, the disinfecting cap comprising:
a housing defining an inner cavity, the housing comprising:
a top wall;
a sidewall extending down from the top wall, the sidewall comprising a threaded inner surface configured to interlock with a mating feature of the luer hub;
an open bottom end formed by the sidewall and defining an opening configured to receive the luer hub; and
a tapered protrusion extending downward from the top wall, with the top wall, the sidewall, and the tapered protrusion defining the inner cavity; and
an annular-shaped absorbent disinfecting member disposed within the housing adjacent an inner surface of the top wall and positioned about the tapered protrusion, the absorbent disinfecting member including a disinfecting solution configured to disinfect portions of the luer hub;
wherein the tapered protrusion is configured to interfit with the luer hub to seal off the IV catheter assembly.

2. The cap of claim 1, wherein the absorbent disinfecting member comprises a compressible sponge or open cell foam, and wherein the sponge or open cell foam is compressed upon engaging of the disinfecting cap with the luer hub, to release at least some of the disinfecting solution upon being compressed.

3. The cap of claim 1, wherein the tapered protrusion has a length less than a length of the sidewall.

4. The cap of claim 1, further comprising a peel-off protective cover positioned over the open bottom end of the housing.

5. The cap of claim 1, wherein the housing comprises a single-molded part formed of a rigid thermoplastic polymer.

6. The cap of claim 1, wherein the tapered protrusion comprises a hollow protrusion.

7. The cap of claim 1, wherein the luer hub comprises a female luer connector comprising a threaded outer surface that engages the threaded inner surface of the sidewall.

8. The cap of claim 7, wherein the disinfecting member is configured to clean and/or disinfect the threaded outer surface of the female luer connector.

9. The cap of claim 7, wherein the female luer connector defines a tapered cavity with which the tapered protrusion is interfit to seal off the IV catheter assembly.

10. An intravenous (IV) catheter assembly comprising:
a catheter hub comprising a distal end and a proximal end;
a catheter coupled to the distal end of the catheter hub and extending out distally therefrom, so as to be positionable intravenously within a patient;
an extension line coupled to the proximal end of the catheter hub;
a luer hub positioned at a proximal end of the extension line, the luer hub in fluid communication with the catheter through the extension line and the catheter hub; and
a disinfecting cap configured to mate with the luer hub, the disinfecting cap comprising:
a housing comprising a top wall, a sidewall extending down from the top wall and having a threaded inner surface, an open bottom end formed by the sidewall and defining an opening configured to receive the luer hub, and a tapered protrusion extending downward from the top wall, with the top wall, the sidewall, and the tapered protrusion defining an inner cavity within the housing; and
an annular shaped absorbent disinfecting member disposed within the housing adjacent an inner surface of the top wall and positioned about the tapered protrusion, the absorbent disinfecting member including a disinfecting solution configured to disinfect portions of the luer hub;
wherein the tapered protrusion is configured to interfit with the luer hub to seal off the IV catheter assembly.

11. The IV catheter assembly of claim 10, wherein the luer hub comprises a female luer connector including:
a hub body comprising a threaded outer surface configured to engage the threaded inner surface of the sidewall; and
a tapered cavity formed in the hub body and configured to mate with the tapered protrusion to seal off the IV catheter assembly.

12. The IV catheter assembly of claim 11, wherein the threaded inner surface of the sidewall comprises a single-start thread, and wherein the threaded outer surface of the female luer connector comprises a double-start thread defining thread two grooves.

13. The IV catheter assembly of claim 12, wherein the single-start thread engages a first thread groove of the threaded outer surface, and wherein a second thread groove of the threaded outer surface remains open, with the second thread groove forming a fluid channel through which the disinfecting solution can flow when the female luer connector is coupled with the disinfecting cap.

14. The IV catheter assembly of claim 11, wherein the threaded inner surface of the sidewall comprises a double-start thread, and wherein the threaded outer surface of the female luer connector comprises a double-start thread.

15. The IV catheter assembly of claim 10, wherein the absorbent disinfecting member comprises a compressible sponge or open cell foam, and wherein the sponge or open cell foam is compressed upon engaging of the disinfecting cap with the luer hub, to release at least some of the disinfecting solution upon being compressed.

16. The IV catheter assembly of claim 10, wherein the disinfecting cap further comprises a peel-off protective cover positioned over the open bottom end of the housing.

17. The IV catheter assembly of claim 16, wherein a bottom edge of the sidewall comprises a flange formed thereon, with the peel-off protective cover sealed on the flange.

18. The IV catheter assembly of claim 10, wherein the tapered protrusion has a length less than a length of the sidewall, such that the tapered protrusion is vertically spaced apart from the open bottom end.

19. The IV catheter assembly of claim 10, wherein the tapered protrusion extends to a bottom edge of the sidewall, such that the tapered protrusion is flush with the open bottom end.

20. The IV catheter assembly of claim 17, wherein the tapered protrusion comprises a hollow protrusion.
